(19) United States
(12) Patent Application Publication
(10) Pub. No.: US 2022/0181891 A1
(43) Pub. Date: Jun. 9, 2022

(54) ULTRASOUND DRIVEN MXENE HYDROGEL ELECTRICAL POWER GENERATOR AND METHOD

(71) Applicant: KING ABDULLAH UNIVERSITY OF SCIENCE AND TECHNOLOGY, Thuwal (SA)

(72) Inventors: Kang Hyuck LEE, Thuwal (SA); Yizhou ZHANG, Thuwal (SA); Husam Niman ALSHAREEF, Garland, TX (US)

(21) Appl. No.: 17/605,728

(22) PCT Filed: May 4, 2020
(86) PCT No.: PCT/IB2020/054223

§ 371 Date: Oct. 22, 2021

Related U.S. Application Data
(60) Provisional application No. 62/843,743, filed on May 6, 2019.

(51) Int. Cl. H02J 7/00; A61M 5/142; A61B 5/00; A61L 24/00; A61L 24/02; C01B 32/921

(52) U.S. Cl. CPC H02J 7/0042; A61M 5/14248; A61B 5/6832; A61L 24/0031; C01P 2004/20; C01B 32/921; A61M 2205/825; A61B 2560/0214; C01P 2006/40; A61L 24/02

(57) ABSTRACT

An electrical power generator includes an M-gel layer that includes MXene and a hydrogel, first and second flexible layers that sandwich the M-gel layer so that the M-gel layer is sealed from an ambient, and a single terminal electrically connected to the M-gel layer. The M-gel layer is configured to transform acoustic energy into electrical energy.

### CROSS-REFERENCE TO RELATED APPLICATIONS

This application claims priority to U.S. Provisional Patent Application No. 62/843,743, filed on May 6, 2019, entitled “ULTRASOUND DRIVEN MXENE HYDROGEL ELECTRICAL POWER GENERATOR FOR IMPLANTABLE BATTERIES,” the disclosure of which is incorporated herein by reference in its entirety.

### BACKGROUND

### Technical Field

Embodiments of the subject matter disclosed herein generally relate to an electrical power generator and method for generating electrical energy from acoustic energy, and more particularly, to an MXene Hydrogel based electrical power generator that is configured to transform ultrasound energy into electrical energy.

### Discussion Of The Background

Conventional drug delivery routes provide limited control over the spatial and temporal resolution of the drug release. Often, the desired availability of the therapeutic drug in the target site can only be achieved by either increasing the dose volume or the dosing frequency, both of which are undesired due to possible mis-dosage.

With the advance of semiconductor devices and increased miniaturization of devices that use such semiconductor devices, many applications are targeting the human body and the specific field of supplying a desired drug to the human body, or collecting information about the human body, or supplying an electrical current to the human body. However, as shown in FIG. 1, all these miniaturized semiconductor devices 100 that are implanted into the human body 102, for example, insulin pump, pacemaker, etc., require an energy source 104. Traditionally, the energy source for such devices is a small battery that is recharged, from time to time, by induction, i.e., by placing an electromagnetic coil 110 outside the body, and connecting the battery 104 to a corresponding electromagnetic coil 106 inside the body 102. The energy 112 (illustrated by a magnetic field) generated by the outside coil 110 is transmitted by induction to the inside coil 106 and thus, the inside coil 106 can recharge the battery 104.

However, the induction effect generates heat, which can make uncomfortable the subject wearing the semiconductor device 100. Further, the subject has to remember every day or at a given time, to bring the external coil 110 next to the internal coil 106, to recharge the battery 106.

Thus, there is a need for finding other means for recharging a battery associated with a semiconductor device that is implanted into the human body, or has no available energy source for recharging.

### BRIEF SUMMARY OF THE INVENTION

According to an embodiment, there is an electrical power generator that includes an M-gel layer that includes MXene and a hydrogel, first and second flexible layers that sandwich the M-gel layer so that the M-gel layer is sealed from an ambient, and a single terminal electrically connected to the M-gel layer. The M-gel layer (202) is configured to transform acoustic energy into electrical energy.

According to another embodiment, there is a recharging system that includes a medical device that includes a battery, the medical device being configured to monitor a parameter of a human body or for delivering a substance to the human body, and an electrical power generator for recharging the battery with electrical energy. The electrical power generator includes an M-gel layer that includes MXene and a hydrogel, first and second flexible layers that sandwich the M-gel layer so that the M-gel layer is sealed from an ambient, and a single terminal electrically connected to the M-gel layer. The M-gel layer is configured to transform acoustic energy into the electrical energy.

According to still another embodiment, there is a method for recharging a medical device that includes a battery. The method includes using the medical device to monitor a parameter of a human body or to deliver a substance to the human body, harvesting with an electrical power generator acoustic energy, transforming the acoustic energy into electrical energy with an electrical power generator that is located inside the human body, wherein the electrical power generator includes an M-gel layer that includes MXene and a hydrogel, first and second flexible layers that sandwich the M-gel layer so that the M-gel layer is sealed from an ambient, and a single terminal electrically connected to the M-gel layer, and re-charging the battery of the medical device with the electrical energy generated the electrical power generator.

### BRIEF DESCRIPTION OF THE DRAWINGS

Fora more complete understanding of the present invention, reference is now made to the following descriptions taken in conjunction with the accompanying drawings, in which:

FIG. 1 is a schematic diagram of a medical device that is powered by a battery and is attached to the human body;

FIGS. 2A and 2B illustrate an electrical power generator that transforms acoustic energy into electrical energy;

FIG. 3 illustrates strain sensing capabilities of an M-gel material that is used by the electrical power generator;

FIG. 4 illustrates a system that is used for testing the electrical power generator under various conditions;

FIGS. 5A to 5C illustrate the voltage and current characteristics generated by the electrical power generator;

FIGS. 6A to 6C illustrate the voltage generated by the electrical power generator under various media conditions;

FIG. 7 illustrates the change in voltage of the electrical power generator as a function of the amount of hydrogel;

FIGS. 8A to 8C illustrate a configuration of the electrical power generator that takes advantage of tribo-electricity;

FIGS. 9A to 9C illustrate another configuration of the electrical power generator that takes advantage of tribo-electricity;

FIGS. 10A to 10C illustrate various materials that are positive or negative with regard to the tribo-electricity effect;

FIG. 11 illustrates an enhanced electrical power generator that have additional layers for generating tribo-electricity;

FIGS. 12A to 12C illustrate the voltage and current characteristics generated by the enhanced electrical power generator;

FIG. 13 illustrates the change in voltage of the enhanced electrical power generator as a function of a thickness of a medium separating the electrical power generator from an acoustic source;

FIGS. 14A to 14C illustrate an implementation of the electrical power generator within a medical device;

FIG. 15 illustrates the voltage generated by the electrical power generator when embedded in actual flesh;

FIG. 16 illustrates the charging of a capacitor with the electrical power generator when embedded in flesh;

FIG. 17 illustrates a recharging system that uses the electrical power generator to recharge the battery of a medical device; and

FIG. 18 is a flowchart of a method for recharging a battery of a medical device with the electrical power generator.

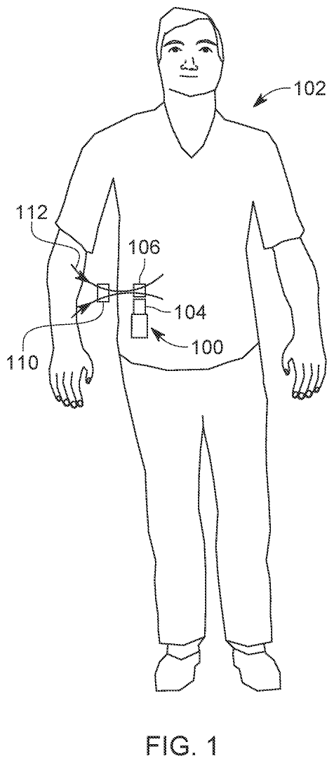

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a drug delivery system that is implanted into the human body for delivering a drug, and has as a source of energy a battery. However, the embodiments to be discussed next are not limited to a drug delivery system, but may be applied to other delivery systems or monitoring systems, i.e. systems that do not deliver anything.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel energy generating device includes an MXene hydrogel layer sandwiched between two flexible layers. Acoustic energy, either from the ambient or generated on purpose, interacts with the MXene hydrogel layer. The MXene hydrogel layer transforms the acoustic energy (especially ultrasound energy) into electrical energy, which is harvested and supplied to a battery for recharging the battery.

In this regard, the acoustic energy is a ubiquitous, clean and sustainable energy resource. The major approach to utilize it is to harvest ultrasonic power based on mechanisms such as piezoelectric and tribo-electric effects. However, it requires specific materials and carefully designed complex device geometries that can cause enough mechanical displacement of the active materials to convert the acoustic energy into the electric energy. Therefore, traditional ultrasonic transducers based on piezoelectric or tribo-electric effects are insufficient for miniaturized, flexible and implantable device applications.

Moreover, when an ultrasound wave passes through more than two media, a part of its energy is consumed because of not only the acoustic impedance, but also the reflection between the media. Reflectance of the ultrasound wave when passing through two different media increases as the difference of acoustic impedance between the media is increased (the high density materials have higher acoustic impedance than the low density materials). Therefore, it is necessary to match the various media with similar density to reduce the reflection of the ultrasound waves.

According to an embodiment, it is possible to harvest the acoustic energy by using an electrical power generator, which is based on the electroacoustic phenomena of the MXene hydrogel material (called herein M-gel layer) under compressional ultrasound waves. This electrical power generator that uses the M-gel layer is called herein the “M-gel generator.” As discussed later, the output performance of this electrical power generator is improved by coupling it with tribo-electric active materials. Moreover, the M-gel generator can be used in applications such as quick charging of a capacitor, and powering implantable devices through skin.

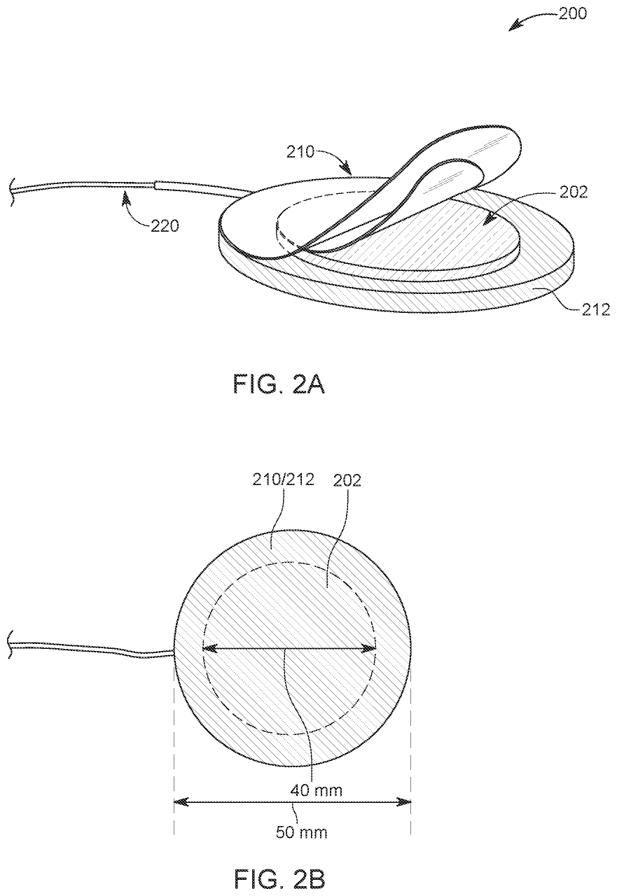

As shown in FIGS. 2A and 2B, an M-gel generator 200 includes a layer 202 of M-gel material having a given thickness (e.g., 1 mm, but can be up to 1 cm). The M-gel layer 202 is sandwiched between two flexible layers 210 and 212, and is connected to a grounded terminal of an oscilloscope (the internal impedance of the probe may be 10 MΩ) through a single terminal 220, which may be a coated copper wire. In one application, the two flexible layers fully insulate or seal the M-gel layer from the ambient. In this application, only the single terminal 220 is electrically connected to the M-gel layer, i.e., there is no other terminal connected to the M-gel layer. The M-gel layer 202 may be shaped in a circular fashion and may have a diameter of about 40 mm while the flexible layers 210 and 212 may have a size of about 50 nm. Smaller or larger sizes for these layers may be used. Also, other shapes may be used for these layers.

In one embodiment, the flexible layers have a surface area larger than the M-gel layer 202 so that the two flexible layers can fully encapsulate the M-gel layer. In one application, the two flexible layers are fully connected to their circumferences (or perimeters) to form an internal chamber to receive and fully seal the M-gel layer inside the formed chamber. In this way, no humidity or any other chemical may enter the chamber and the M-gel layer. The two flexible layers may be formed from any material that is easily bendable so that the exterior acoustic energy can deform them, so that these layers directly act on the M-gel layer and also easily conform to parts of the human body. For example, in one embodiment, the two layers may be formed from an eco-flex material, e.g., silicone rubber or a polymer. The material for the two flexible layers is also selected to not be toxic to the human body, and not to release or leak any dangerous components into the human body.

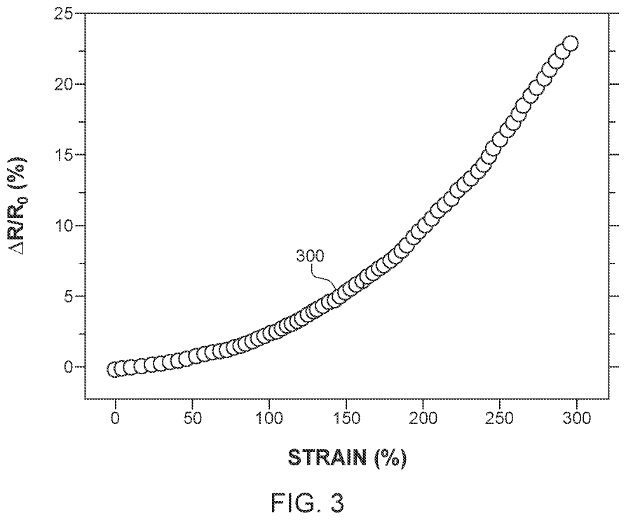

The M-gel layer 202 may include a polymer composite material that is very malleable. When strain is applied to the polymer composite, its fractional resistance changes (henceforth referred to as ΔR/R0, where R0 is the resistance of the sensing material without strain and ΔR is the amount of resistance change after applying strain) according to curve 300 shown in FIG. 3, as a function of the applied strain (%). This means that the polymer composite material can be used for all the typical strain sensor applications. Moreover, this polymer composite material can fit arbitrary surfaces with complex shapes such as human skin near the joints, or it can fully cover the tiny spaces between wrinkles due to the sticky and easy shape-transformable characteristics of its components.

According to an embodiment, this polymer composite material includes (1) a viscoelastic hydrogel and (2) conductive nanofillers. The hydrogel is a class of viscoelastic materials composed of three-dimensional (3D) networks of hydrophilic polymers crosslinked, chemically or physically, with the capacity to absorb and retain a large amount of water (up to 90%). The hydrogel may include natural or synthetic polymeric networks. The hydrogels also possess a degree of flexibility very similar to natural tissue, due to their significant water content. The term “viscoelastic” is understood herein to be a property of materials to exhibit both viscous and elastic characteristics when undergoing deformation. Viscous materials resist shear flow and strain linearly with time when a stress is applied. Elastic materials strain when stretched and quickly return to their original state once the stress is removed. Viscoelastic materials have elements of both of these properties and, as such, exhibit time-dependent strain.

Conductive hydrogels are potential candidates for electro-mechanical sensing materials for applications such as wearable electronics, soft robotics and prosthetics, which require the sensors to be highly sensitive, stretchable, and easily adhere to arbitrary and complex surfaces, such as human skin. However, hydrogel based electro-mechanical sensors generally exhibit relatively low sensitivity. Furthermore, as viscoelastic materials, their electro-mechanical responses to external forces show unstable noises with hysteresis and fluctuation due to the unexpected viscous deformations.

Recently, a special class of conductive nanofillers, whose geometry and conductivity can both be changed by deformations, have been used to improve the sensitivity of composite strain sensors (see, for example, Wang et al., Rev. Sci. Instrum. 84, (2013) 105005, Lin et al., J. Mater. Chem. C, 4, (2016) 6345-6352, Tang et al., ACS Appl. Mater. Interfaces 7, (2015) 27432-27439, and Boland et al., Science 354, (2016) 1257-1260). Although these nanofiller networks can greatly improve the sensitivity, they still cannot overcome the limitations originated from viscous deformations. In addition, these nanofillers usually cannot maintain their deformed network structure caused by viscous deformations, due to their easy motion within the hydrogel matrix and possible rearrangement of the network structure by an electric field. As a result, their sensing reliability is compromised.

The inventors of the present application have realized that MXenes, a class of two-dimensional (2D) early transition metal carbides and/or carbonitrides and/or nitrides, might cure the problems noted in the above nanofillers. These materials consist of few atoms thick layers of transition metal carbides, nitrides or carbonitrides. More specifically, a MXene includes (1) a transition metal M and (2) atom X, which can be C or N based. The MXenes may also include a functional group T, which may include O, F or OH. The MXenes may be generically represented as Mn+1Xn, when n is 1, 2 or 3. MXenes combine the metallic conductivity of the transition metal carbides and hydrophilic nature because of their hydroxyl or oxygen terminated surface.

The MXenes may be prepared by selectively etching out the element “A” from the three-dimensional (3D) structured ceramic known as MAX. Similar to graphene, these 2D laminated nanocrystals exhibit large specific surface area, high electrical conductivity, favorable mechanical strength and other interesting characteristics. Owing to the abundant surface function groups T (OH, O, F, H, etc.), MXenes show good hydrophilicity. Because of the multiple active sites on the surface, MXenes can be used as active materials for catalysis, electrochemical energy storage, etc.

According to an embodiment, a method for preparing MXene based hydrogel (called M-hydrogel in here) is now discussed. Note that the M-hydrogel now discussed is only one possible implementation of the polymer composite material discussed above. In a first step, MXene (Ti₃C₂) nanosheets are provided in a mixing bowl. Next, a commercial low-cost hydrogel (“crystal clay,” which is composed of Poly (vinyl alcohol), water and anti-dehydration additives) is also provided in the mixing bowl. Next, the two components are mixed together to form the M-hydrogel. In one application, the nanosheets are randomly distributed inside the hydrogel. The concentration of the Mxene by mass weight may be larger than zero and smaller than 10%. The resulting M-hydrogel shows extremely high-strechability, which is much higher than the pristine hydrogel, and malleability. These properties are attributed to the abundant hydrophilic surface functional groups such as —F and —OH on MXene nanosheets, which form strong hydrogen bonds between the MXene nanosheets and the hydrogel matrix.

Thus, unlike traditional hydrogels using other conductive fillers, the M-hydrogels is “softer” and more stretchable than the pristine hydrogel. The strong hydrogen bonding and the half-liquid property of hydrogel also endow the M-hydrogel with instantaneous self-healability (i.e., the object can restore its original properties after plastic and/or viscous deformations), and an excellent conformability and adhesiveness to various surfaces including the human skin.

It was observed that the addition of MXene nanosheets to the hydrogel may significantly improve the tensile strain sensitivity of the hydrogel, e.g., 5 times increase at 4.1 wt % of the MXene nanosheets. At the same time, the hydrogel shows a much higher sensitivity under compressive strains.

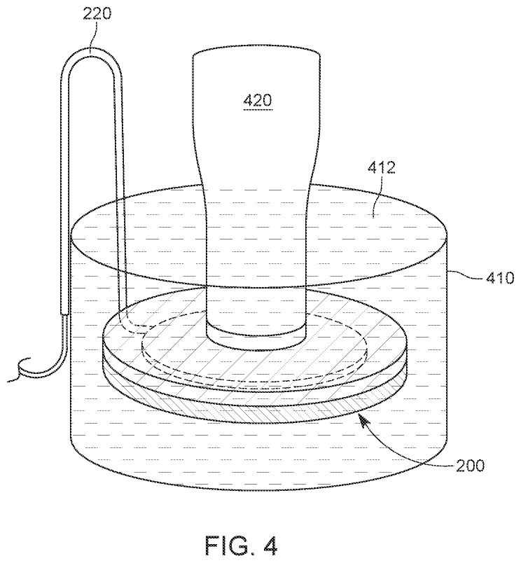

Returning to the M-gel generator 200, FIG. 4 shows it placed in a container 410 holding water 412 and a sonicator 420 being placed on top of the M-gel generator 200. The sonicator 420 is used to generate ultrasound waves for testing the response of the M-gel layer 202 to the ultrasound waves. In this configuration, the ultrasound waves propagate to the M-gel generator across the water medium 412, which has a 2 mm thickness from the tip of the sonicator 420. The frequency of the generated ultrasound waves is 20 KHz in this embodiment and the ultrasound waves were turned on at constant time intervals to clearly distinguish the on/off states.

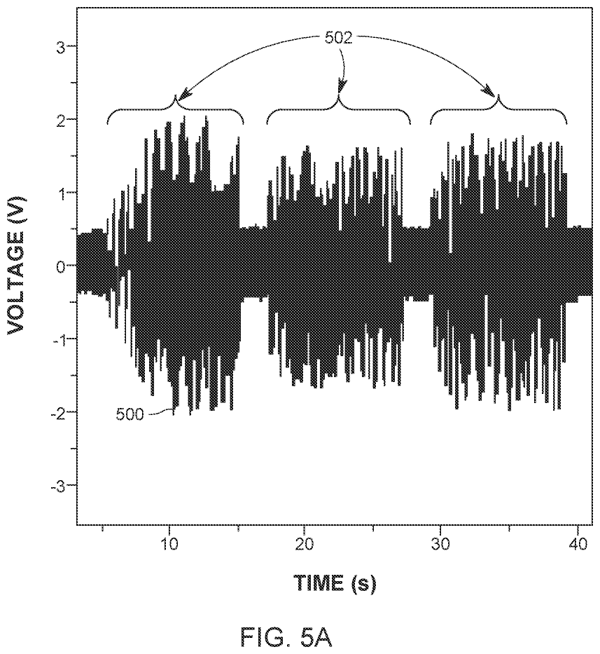

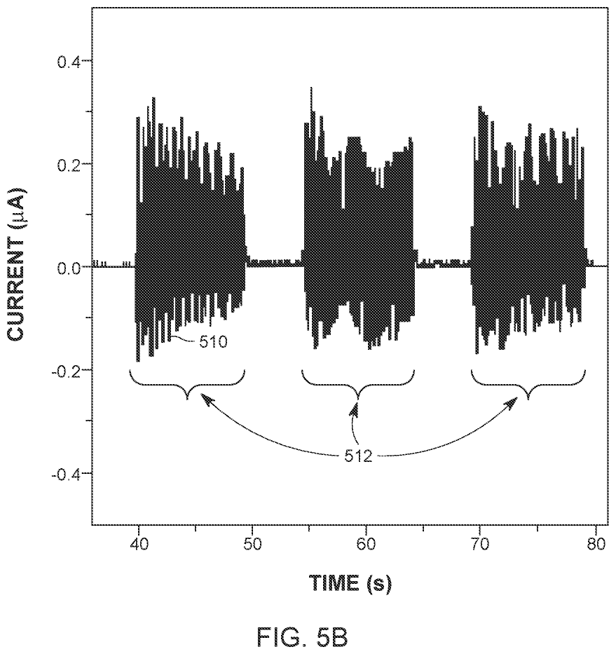

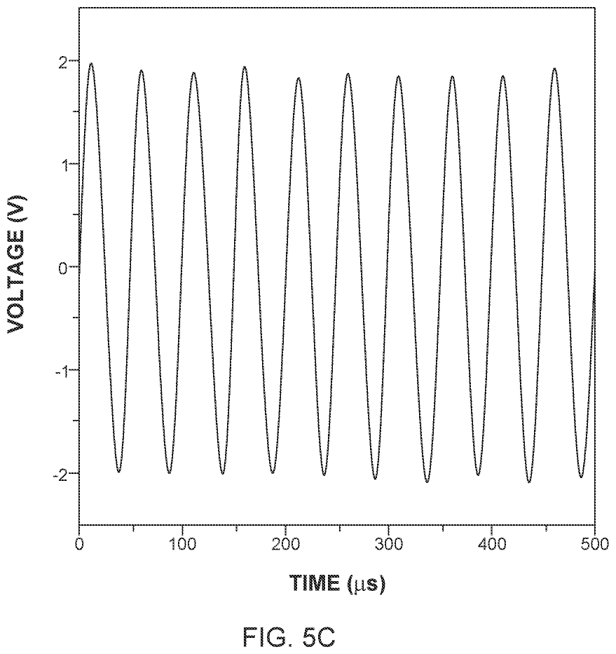

It was found that the M-gel generator 200 generates an alternating voltage 500, during the on periods 502, as shown in FIG. 5A and an alternating current 510, during the on periods 512, as shown in FIG. 5B, as the ultrasound waves propagate through the M-gel layer 202. The peak to peak output voltage and short circuit current are ˜4V and ˜0.5 uA, respectively. The output voltage of the M-gel generator exhibits a sine waveform with a frequency of 20 kHz (see FIG. 5C, which plots the voltage versus time as measured by the oscilloscope at the micro-second time scale), which perfectly matches the ultrasound waveform generated by the sonicator 420. It is believed that the AC voltage generated by the M-gel generator 200, from the ultrasound energy, is due to the streaming vibration potential (SVP), which refers to an electric signal that arises when an acoustic wave propagates through a porous body (Mxene) in which the pores are filled with fluid (hydrogel). In this case, the M-gel layer 202 is filled with a fluid (that may include water among other liquids) as discussed above with regard to the method of making the M-gel material. The incident and reflected acoustic waves superimpose to cause an oscillatory fluid motion in the plane of the interface, thereby generating the AC streaming potential at the same frequency as the sound waves.

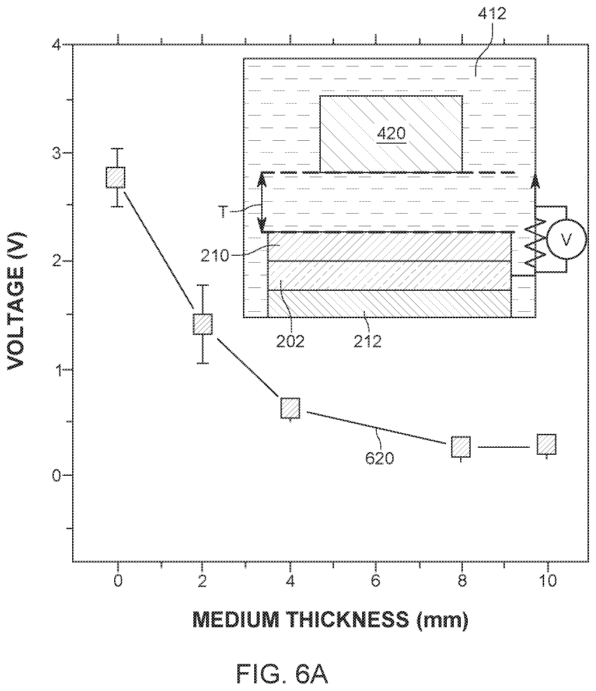

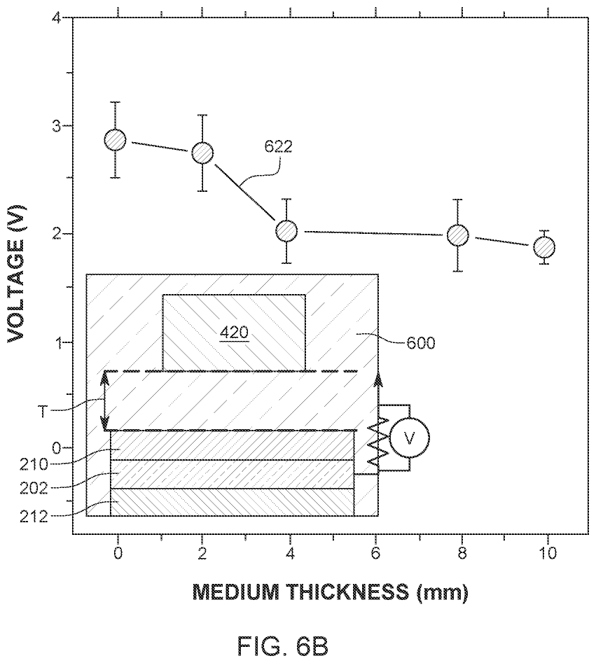

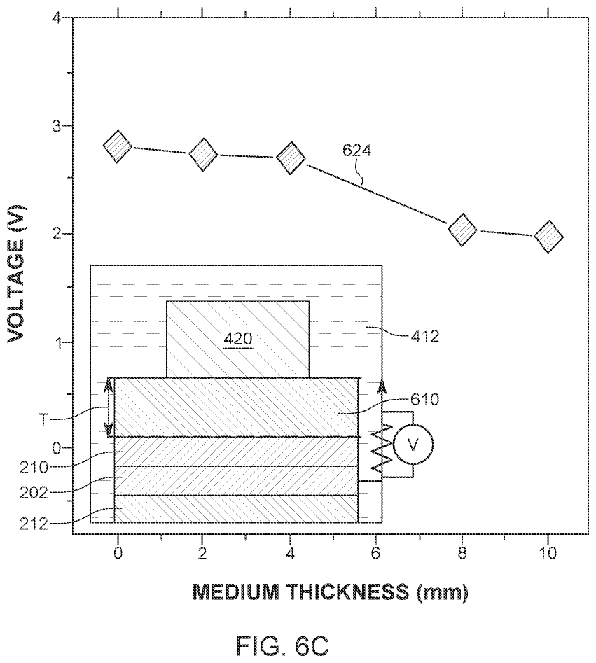

For the configuration shown in FIG. 4, until the ultrasound waves reach the M-gel layer 202, they have to travel through the water 412 and the two flexible layers 210 and 212. If the voltage generation of the M-gel layer 202 in the generator 200 is originated from the ultrasound waves generated by the sonicator 420, it should show dependence on (a) the kind of the medium present between the sonicator and the M-gel device, and (b) the thickness of the medium. Therefore, the inventors have measured the output voltages of the M-gel generator 200 when placed in water 412, hydrogel 600, and eco-flex medium 610, respectively, as shown in the insets of FIGS. 6A to 6C, respectively. In this embodiment, both the thickness of the M-gel layer 202 and the thickness of the first and second flexible layers 210 and 212 is 1 mm. The layer thickness for the water 412, hydrogel 600, and eco-flex material 610 placed between the tip of the sonicator and the surface of the M-gel generator 200 is T.

If the thickness T of the medium is zero, which means that the ultrasonic-tip is in direct contact to the top surface of the M-gel generator 200, the output voltages with the water, hydrogel, and eco-flex medium were almost the same, at around 2.8 V, as shown by curves 620 to 624 in FIGS. 6A to 6C. The voltage was reduced to 0.37V, 1.78V, and 2.14V when the thickness T of the water 412, hydrogel 600, and eco-flex medium 610 was increased to 8 mm respectively, as also shown in FIGS. 6A to 6C. The voltage reduction behavior of the M-gel generator 200 in different media resembles the behavior of the reflectance of the ultrasound wave, which increases when passing through the media between the sonicator and the device. This suggests that the M-gel generator can effectively convert the ultrasound energy into electrical energy in the presence of a medium, and the possibility of optimizing the conversion performance by choosing a suitable medium.

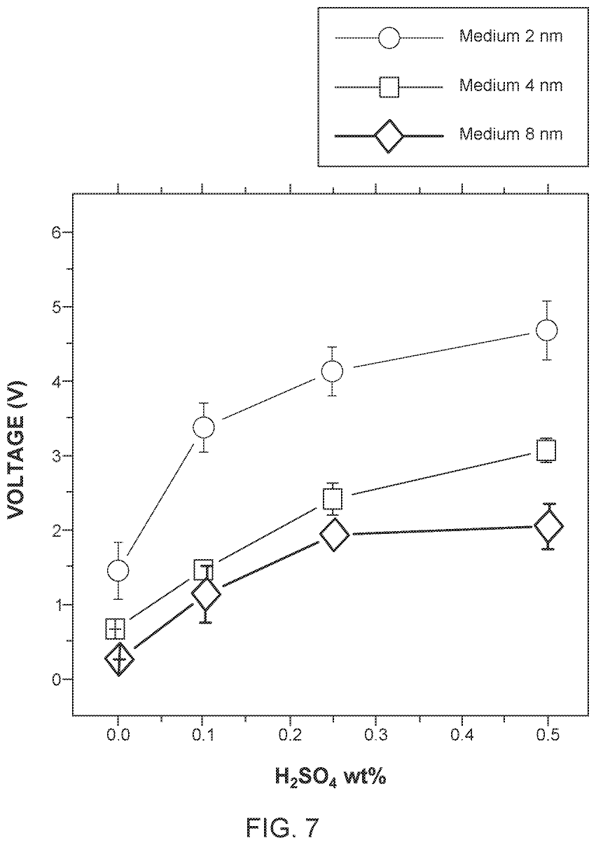

The inventors also found that the electrical output of the M-gel generator can be improved by changing the electrolytic state of the M-gel layer 202, by adding 0.1˜0.5 wt % of H₂SO₄ into the hydrogel of the M-gel layer. The output voltage of such M-gel layer almost quadrupled (see FIG. 7) for every studied thickness of the medium, where the medium was the eco-flex material used in the configuration shown in FIG. 6C.

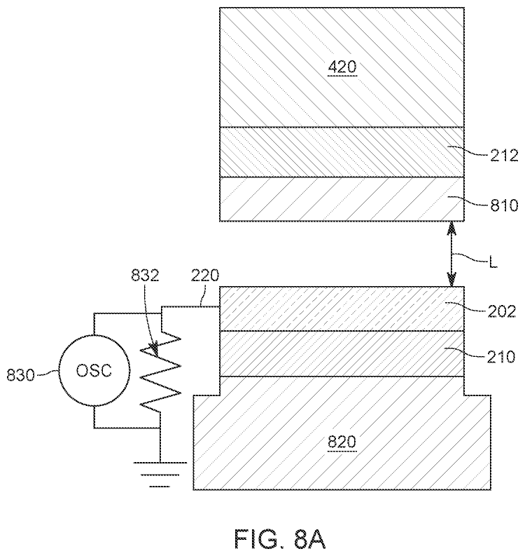

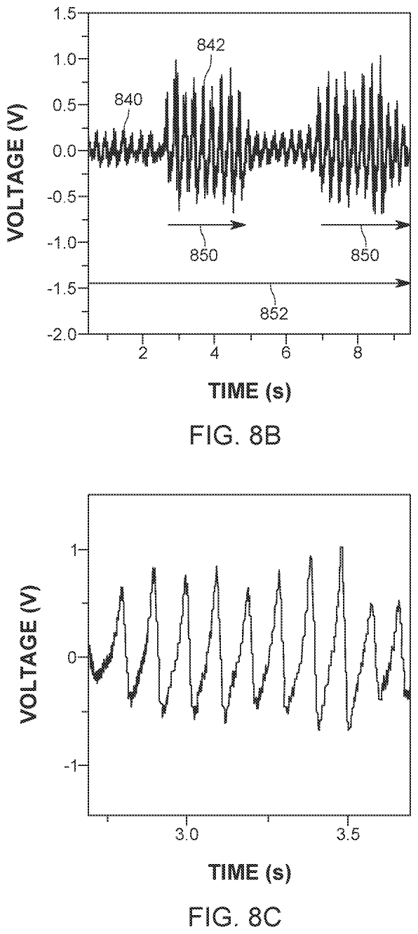

The voltage generation of the M-gel generator 200 can be further improved by harnessing the power of the tribo-electricity effect. To determine the effect of the tribo-electricity on the M-gel generator 200, two configurations were used. The first configuration is illustrated in FIG. 8A, and includes the M-gel layer 202, the first flexible layer 210 placed under and in direct contact to the M-gel layer 202, the second flexible layer 212, which is separated from the M-gel layer 202 by a given gap having a length L, and a nylon layer 810 placed between the second flexible layer 212 and the M-gel layer 202. The sonicator 420 is placed in direct contact with the second flexible layer 212 while a mechanical shaker 820 is placed in direct contact with the first flexible layer 210. The mechanical shaker 820 is added to induce tribo-electricity between the nylon layer 810 and the M-gel layer 202, by moving the nylon layer towards and away from the M-gel layer. In this regard, it is known that the tribo-electricity is related to generating static electrical charges on two materials when they are contacted and separated. For this reason, the shaker 820 moves the M-gel layer 202 over the length L towards and away from the nylon layer 810. The M-gel layer 202 is electrically connected to an oscilloscope 830, which is grounded.

The oscillation of the grounded M-gel layer 202 due to the shaker (20 Hz) generates a small AC voltage 840, as shown in FIG. 8B, which increases rapidly when applying ultrasound waves to the second flexible layer 212 and the nylon layer 810, as illustrated by reference number 842. Arrows 850 indicate in FIG. 8B the time interval that the sonicator 420 is on while arrow 852 indicates the time interval that the shaker 820 is on. When a charged object (nylon layer 810) is approaching the grounded conductor 220, which is also connected to a load 832, and then is moving away from it, a voltage fluctuation is generated by the electrostatic induction. Generally, the surface of the Nylon layer 810 tends to become positively charged by friction and even shows slightly positive-charged surface without a friction while the M-gel layer 202 tends to become negatively charged.

The small voltage fluctuation 840 at the initial stage is due to the electrostatic induction between the slightly charged nylon surface and the M-gel layer. By applying the ultrasound waves to the second flexible layer/Nylon layer, the nylon material becomes more positively charged because ultrasound waves cause tribo-electrification between the second flexible layer and the nylon layer. This tribo-electricity makes larger voltage fluctuations than without ultrasound. The frequency of the induced voltage is similar to the frequency of the shaker, even when applying the ultrasound wave, as shown in FIG. 8C.

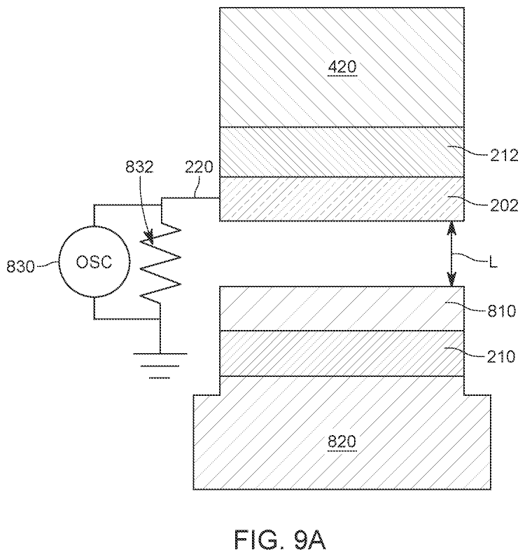

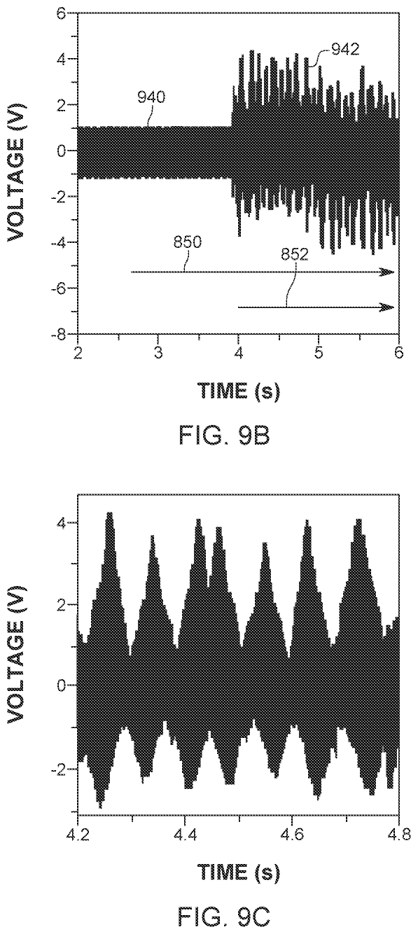

FIG. 9A shows another configuration that has been studied, in which the M-gel layer 202 and the second flexible layer 212 are physically separated by the distance L from the first flexible layer 210 and the nylon layer 810. Further, the shaker 820 is configured to move the first flexible layer 210 and the nylon layer 810 to generate tribo-electric charges. This configuration is used to show the tribo-electric effect on the SVP generation of the M-gel layer 202. When only ultrasound waves are applied by the sonicator 420, the voltage 940 generated by SVP is small, as shown in FIG. 9B, and the voltage has an amplitude of 1V, and is formed in the shape of sine wave with the same frequency as the ultrasound waves. The voltage rapidly increased, see voltage 942, when the surface charged nylon layer 810 (which is pre-charged by friction) repetitively approaches the M-gel layer 202, due to the shaker 820, during the time period 852 when the shaker is on. FIG. 9C shows the voltage generated by the second configuration, which is shown in FIG. 9A, as a function of time, which indicates an AC profile.

The results noted in FIGS. 8B and 8C and also in 9B and 9C indicate that the ultrasound waves can effectively cause tribo-electrification between the first or second flexible layer and the nylon layer, and also that the surface charged layer through tribo-electrification can increase the SVP of the M-gel layer. In other words, it is possible to improve the performance of the M-gel device by using tribo-electrification, which can be achieved, for example, with one or more charged layer (e.g., nylon layer) 810.

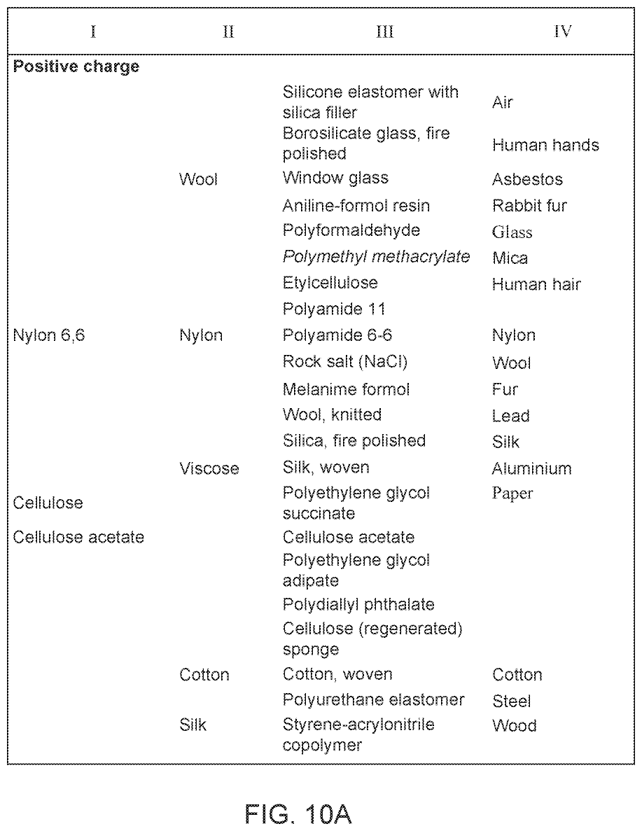

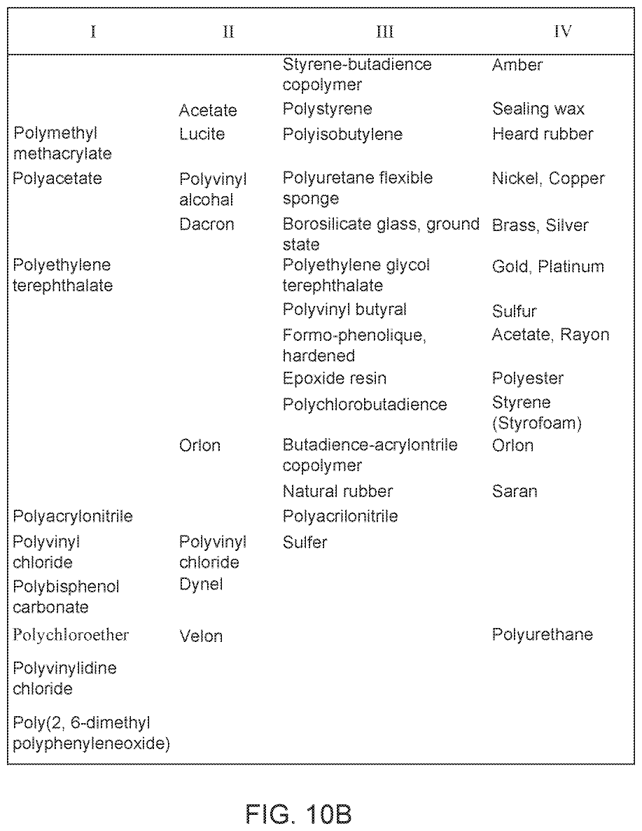

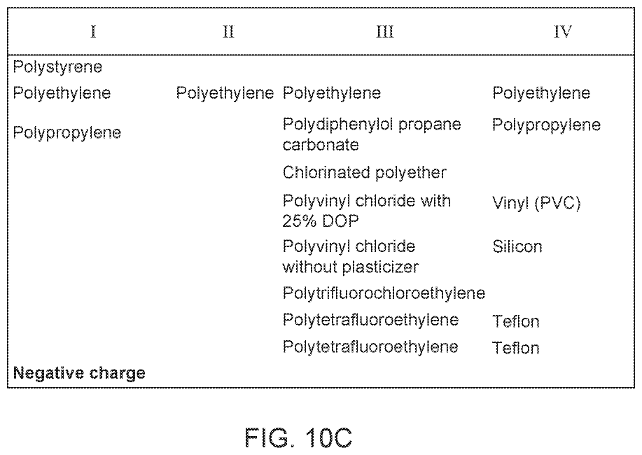

While the embodiments discussed above used a nylon layer 810 for producing the tribo-electricity, it is possible to use other materials for achieving the same effect. In this regard, note that for having a strong tribo-electricity effect, the two layers that generate the static charging are preferably selected from a positive charge material and a negative charge material, so that the electrostatic charges from one material can move to the other material to electrically charge both materials. Thus, in the following, a positive charged material and a negative charged material are preferably selected as components of the M-gel generator 200 that promote the tribo-electricity effect. The positive and negative charged materials have been studied over time and they are generally understood. The table shown in FIGS. 10A to 10C list various such materials arranged in a series, from the most positive charged material to the most negative charged materials. The four columns noted in the table in FIGS. 10A to 10C correspond to four different studies (I to IV) that ordered these materials. Irrespective of the study, it is noted that the nylon material is a positive charged material while the polymers are negative charged materials. This is the reason why in the configurations shown in FIGS. 8A and 9A, the combination of nylon (positive charged material) and first or second flexible layers of a polymer (negative charged material) have been selected. Those skilled in the art would understand that the configurations shown in FIGS. 8A and 9A would also work with other materials from the table of FIGS. 10A to 10C, as long as one material is a positive charged material and the other material is a negative charged material.

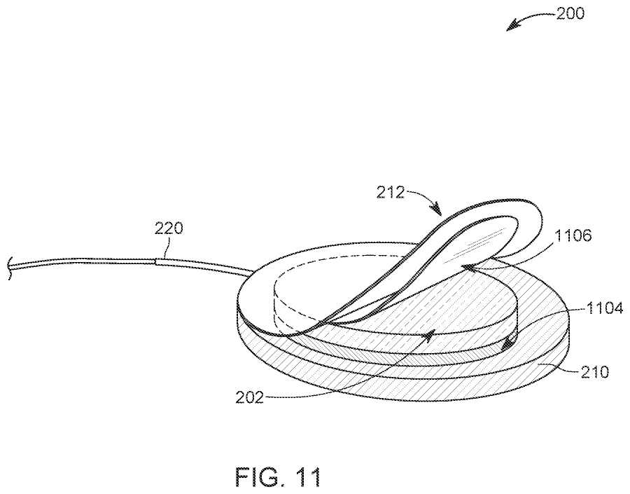

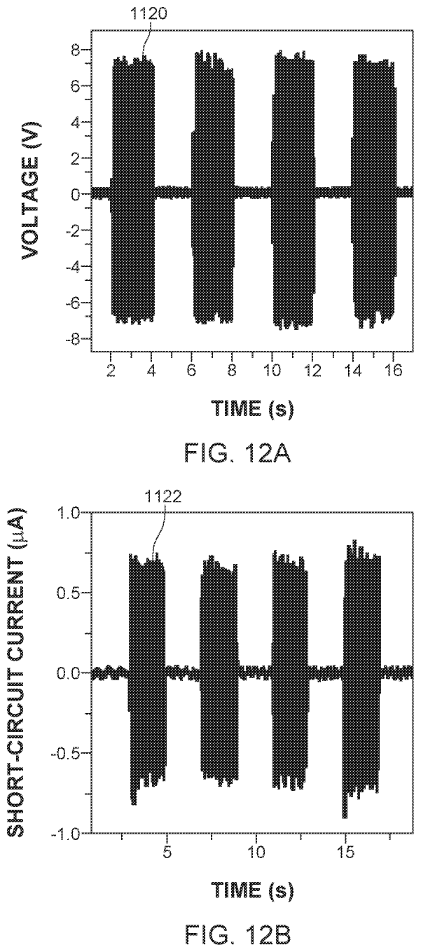

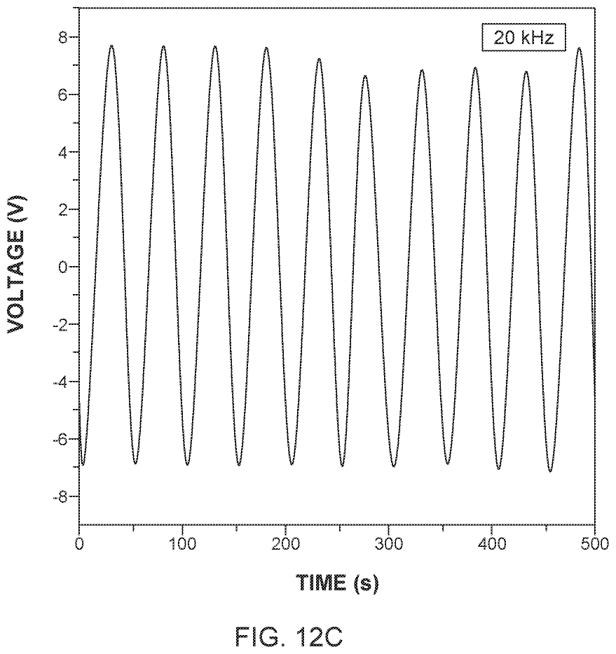

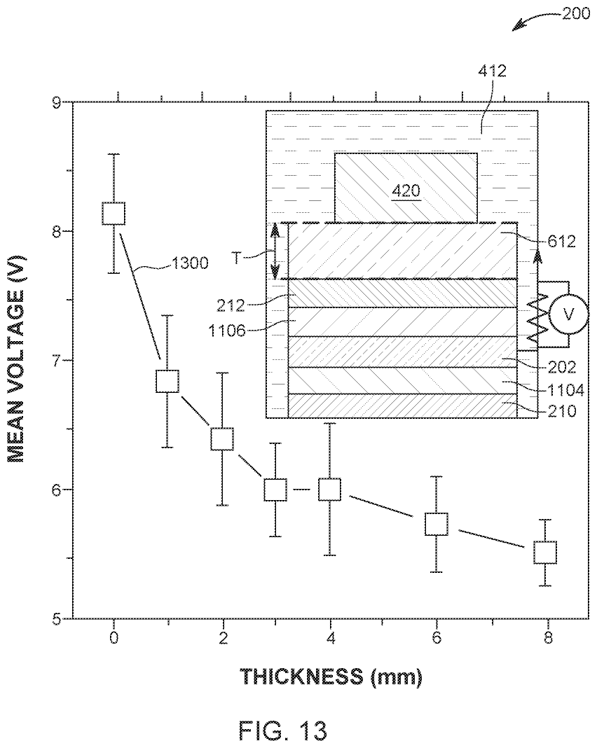

The output power of the M-gel generator 200 can be improved by adding a first nylon membrane 1104 (or other positive charged material from the table in FIGS. 10A to 10C) between the first flexible layer 210 and the M-gel layer 202, and a second nylon membrane 1106, between the second flexible layer 212 and the M-gel layer 202, as shown in FIG. 11A. In one application, only one of the first and second nylon membranes is added to the M-gel generator 200. The addition of these nylon membranes 1104 and 1106 (also called positive charged layers) leads to a four times higher output voltage 1120 (peak to peak voltage is ˜16 V as shown in FIG. 12A) and current 1122 (peak to peak current is ˜1.5 μA as shown in FIG. 12B) values than the M-gel generator 200 without the nylon layers. The output voltage has the same frequency as the applied ultrasound wave, as illustrated in FIG. 12C. The structure of the tribo-electricity enhanced M-gel generator 200 is shown in FIG. 13, together with a thick medium 612 (polymeric material) placed between the sonicator 420 and the top surface of the generator 200. With this configuration and under such conditions, FIG. 13 shows that the voltage 1300 of the enhanced M-gel generator is maintained at above 6V.

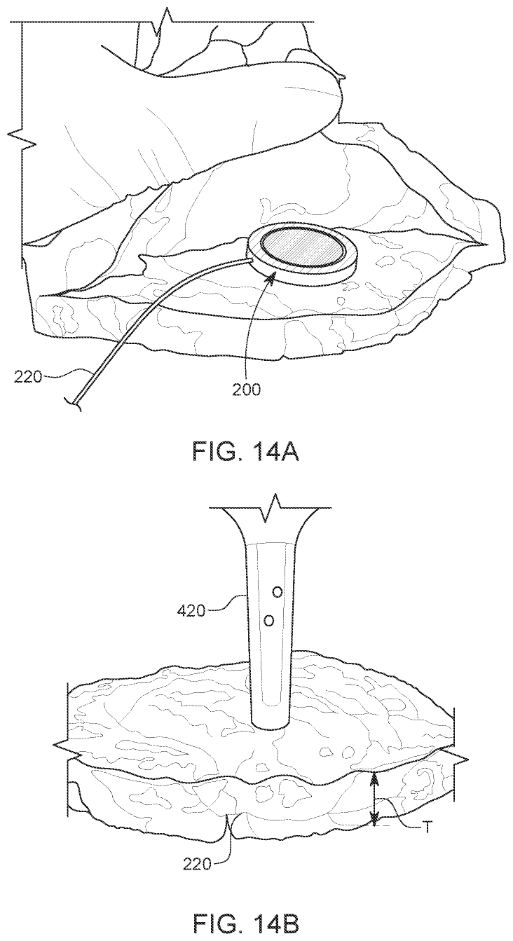

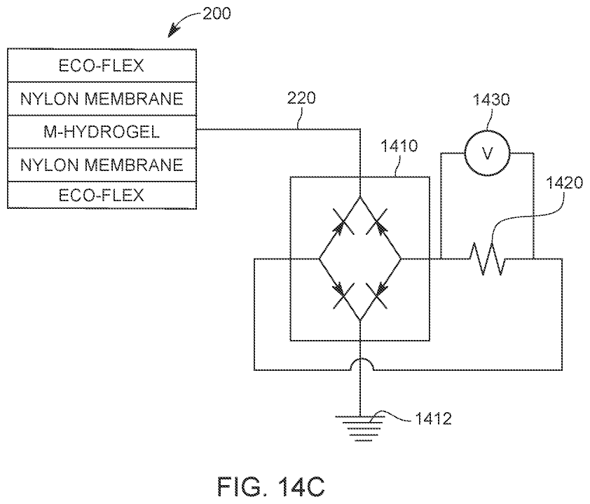

The inventors have tested the feasibility of the M-gel generator 200 for in-vivo applications. For this test, the inventors inserted the enhanced M-gel generator 200 within a piece of fresh beef, as shown in FIG. 14A and 14B, with a comparable thickness relevant for human implantable devices. The thickness T of the beef is about 1.6 cm, the M-gel generator 200 has been placed half way through the thickness of the meet as shown in FIG. 14A, and the tip of the sonicator 420 was placed on top of the beef, as shown in FIG. 14B. A rectifier circuit 1410 is connected between the lead 220 of the generator 200 and the ground 1412, as illustrated in FIG. 14C. Any rectifier circuit 1410 may be used. For simplicity, FIG. 14C shows a bridge rectifier that includes four diodes. The rectifier circuit 1410 is connected to a load 1420 (for example, a resistance or a battery). In one application, the load 1420 is a medical device. The medical device may be a pump that is attached to the human body and configured to deliver a drug. In another application, the medical device is a sensor that monitors a parameter of the body, for example, temperature, blood oxygen, blood sugar, etc. Any medical device that needs a source of electrical power may be connected to the generator 200.

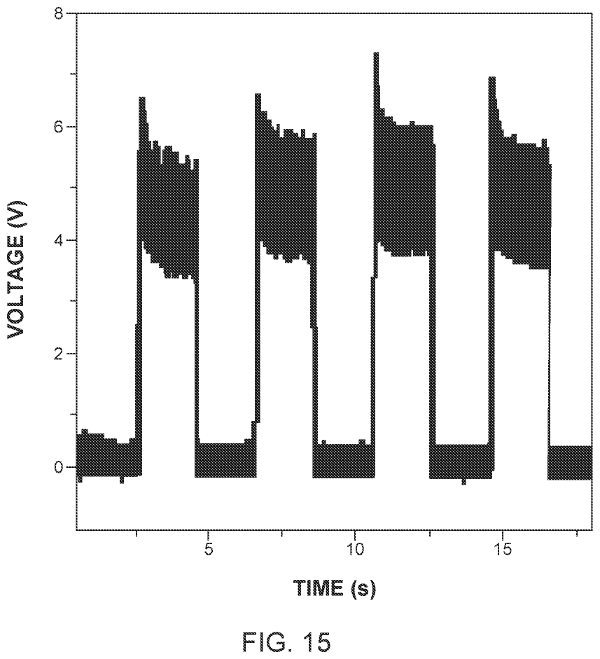

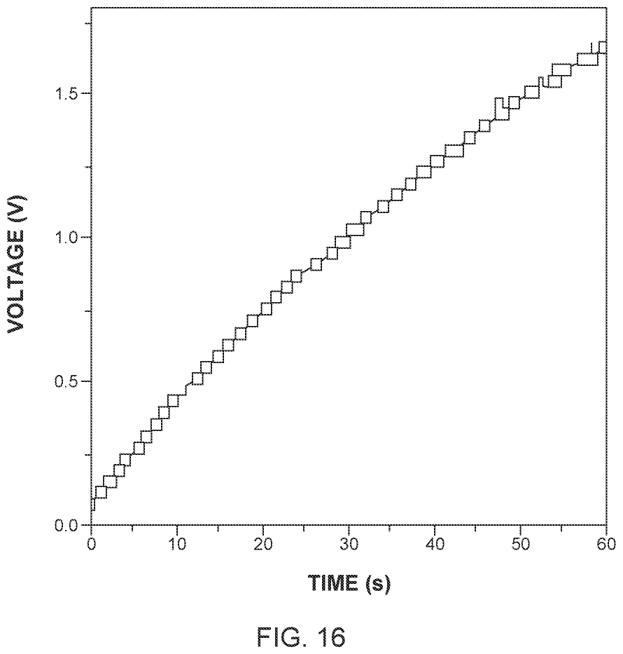

For experimental purposes, a voltage measuring device 1430 is connected across the load 1420 for monitoring the voltage generated by the generator 200. The rectified voltage is maintained above 6V, as shown in FIG. 15, even when the M-gel generator 200 is placed inside the beef. When a capacitor (e.g., 100 μF) was connected to the circuit as the load 1420, the voltage of the capacitor increased to 1.68V during a 60 sec time interval, as shown in FIG. 16.

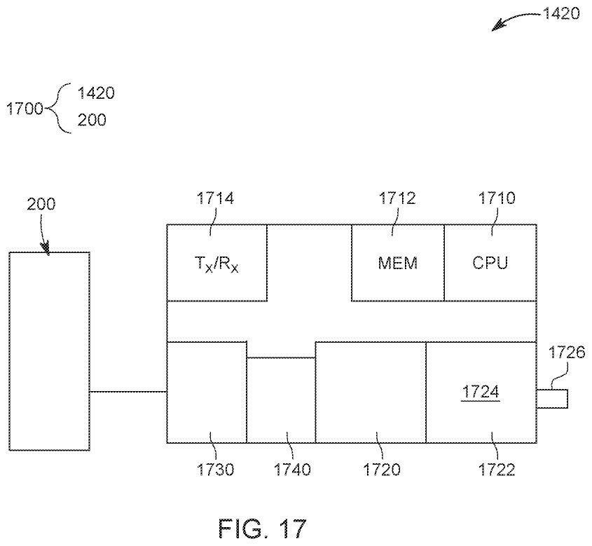

The power delivered from the enhanced M-gel generator can serve as a power source to continuously drive any desired device, for example, the medical device. The medical device 1420, which is schematically shown in FIG. 17 as being part of a recharging system 1700, may include a processor 1710 that communicates with a memory 1712. The processor may communicate with a transceiver 1714 for exchanging data and/or commands. The transceiver 1714 may also communicate with an external device (not shown), for example, a computer or handheld device which is operated by a medical professional or the patient. These persons can then control the medical device 1420, for example, regulating the amount of drug 1724 that is delivered by a pump 1720, from a reservoir 1722, into the human body, for example, through an outlet 1726 (e.g., a needle). The medical device 1420 may be implemented for other purposes into the human body, for example, as a pacemaker, heart monitoring device, brain monitoring device, etc. The medical device 1420 may also include one or more sensors 1740 for measuring one or more parameters of the body, for example, temperature, sugar in blood, etc. The sensor 1740 communicates with the processor 1710 and is supplied with power by a power source 1730. All this information may be stored in the memory 1712.

The medical device 1420 may also include the power source 1730, for example, a battery. The power source 1730 supplies all the above noted components of the medical device with electrical energy. The power source may be electrically connected to the M-gel generator 200 discussed above, for being able to be recharged when desired. For this operation, either ambient ultrasound waves or ultrasound waves produced on purpose, for example, with the sonicator 420, are transformed into electrical energy by the M-gel generator 200.

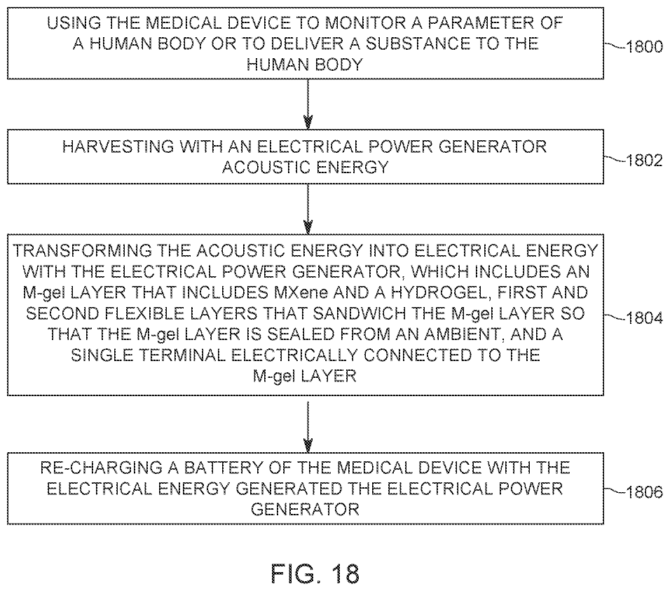

According to an embodiment, which is illustrated in FIG. 18, there is a method for recharging the medical device 1420, which includes a battery 1730. The method includes a step 1800 of using the medical device 1420 to monitor a parameter of a human body or to deliver a substance to the human body, a step 1802 of harvesting with the electrical power generator 200 acoustic energy, a step 1804 of transforming the acoustic energy into electrical energy with the electrical power generator 200, which includes the M-gel layer 202 that includes MXene and a hydrogel, the first and second flexible layers 210, 212 that sandwich the M-gel layer 202 so that the M-gel layer 202 is sealed from an ambient, and a single terminal 220 electrically connected to the M-gel layer 202, and a step 1806 of re-charging a battery 1730 of the medical device 1420 with the electrical energy generated the electrical power generator 200.

The disclosed embodiments provide an electrical power generator which uses ultrasonic energy for generating electrical power. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. An electrical power generator comprising:
an M-gel layer that includes MXene and a hydrogel;
first and second flexible layers that sandwich the M-gel layer so that the M-gel layer is sealed from an ambient; and
a single terminal electrically connected to the M-gel layer,
wherein the M-gel layer is configured to transform acoustic energy into electrical energy.

2. The electrical power generator of claim 1, wherein the M-gel layer transforms ultrasound energy.

3. The electrical power generator of claim 1, wherein the first and second flexible layers include a polymer.

4. The electrical power generator of claim 1, wherein a thickness of the M-gel layer is smaller than 1 cm.

5. The electrical power generator of claim 1, wherein a thickness of the M-gel layer is about 1 mm.

6. The electrical power generator of claim 5, wherein a thickness of each of the first and second flexible layers is about 1 mm.

7. The electrical power generator of claim 1, wherein each of the first and second flexible layers are directly in contact with the M-gel layer.

8. The electrical power generator of claim 1, further comprising:
first and second positive charged layers,
wherein the first positive charged layer is located between the M-gel layer and the first flexible layer, and
wherein the second positive charged layer is located between the M-gel layer and the second flexible layer.

9. The electrical power generator of claim 8, wherein the first and second positive charged layers include nylon.

10. The electrical power generator of claim 8, wherein the first and second flexible layers are negative charged layers.

11. The electrical power generator of claim 10, wherein a first pair of the first flexible layer and the first positive charged layer, and a second pair of the second flexible layer and the second positive charged layer generate tribo-electricity.

12. A recharging system comprising:
a medical device that includes a battery, the medical device being configured to monitor a parameter of a human body or for delivering a substance to the human body; and
an electrical power generator for recharging the battery with electrical energy,
wherein the electrical power generator includes
an M-gel layer that includes MXene and a hydrogel,
first and second flexible layers that sandwich the M-gel layer so that the M-gel layer is sealed from an ambient, and
a single terminal electrically connected to the M-gel layer,
wherein the M-gel layer is configured to transform acoustic energy into the electrical energy.

13. The system of claim 12, wherein the medical device includes a sensor for monitoring a parameter of the human body.

14. The system of claim 12, wherein the medical device includes a pump and a reservoir for storing a drug, and the pump is configured to deliver the drug directly to the human body.

15. The system of claim 12, wherein the first and second flexible layers include a polymer, and wherein a thickness of the M-gel layer is smaller than 1 cm.

16. The system of claim 12, wherein a thickness of the M-gel layer is about 1 mm and a thickness of each of the first and second flexible layers is about 1 mm.

17. The system of claim 12, further comprising:
first and second positive charged layers,
wherein the first positive charged layer is located between the M-gel layer and the first flexible layer, and
wherein the second positive charged layer is located between the M-gel layer and the second flexible layer.

18. The system of claim 17, wherein the first and second flexible layers are negative charged layers.

19. The system of claim 17, wherein a first pair of the first flexible layer and the first positive charged layer, and a second pair of the second flexible layer and the second positive charged layer generated tribo-electricity.

20. A method for recharging a medical device that includes a battery, the method comprising:
using the medical device to monitor a parameter of a human body or to deliver a substance to the human body;
harvesting with an electrical power generator acoustic energy;
transforming the acoustic energy into electrical energy with an electrical power generator that is located inside the human body, wherein the electrical power generator includes an M-gel layer that includes MXene and a hydrogel, first and second flexible layers that sandwich the M-gel layer so that the M-gel layer is sealed from an ambient, and a single terminal electrically connected to the M-gel layer; and
re-charging the battery of the medical device with the electrical energy generated the electrical power generator.
